(19) United States
(12) Patent Application Publication
(10) Pub. No.: US 2019/0223874 A1
(43) Pub. Date: Jul. 25, 2019

(54) LIGATION CLIP WITH TISSUE RETENTION FEATURES

(71) Applicant: Covidien LP, Mansfield, MA (US)

(72) Inventors: Roy Pilletere, North Haven, CT (US); Gregory Morck, Middletown, CT (US); Saumya Banerjee, Hamden, CT (US); Justin Thomas, New Haven, CT (US); Matthew Dinino, Newington, CT (US); Eric Brown, Haddam, CT (US)

(21) Appl. No.: 16/158,368

(22) Filed: Oct. 12, 2018

Related U.S. Application Data
(60) Provisional application No. 62/621,163, filed on Jan. 24, 2018.

(51) Int. Cl. A61B 17/122

(52) U.S. Cl. CPC A61B 17/122; A61B 2017/00429

(57) ABSTRACT

A ligation clip defines a longitudinal axis includes a first jaw, a second jaw, and a hinge portion that pivotably couples the first jaw to the second jaw. The first jaw has a first clamping surface that supports first ribs that defines an angle β with the longitudinal axis. The second jaw has a second clamping surface in opposition to the first clamping surface and supports second ribs that define the angle β with the longitudinal axis. Each of the first and second ribs includes a distal wall and a proximal wall and a wall in opposition to the opposing clamping surface. The distal wall of at least some of the first ribs is positioned in close proximity to the proximal wall of a respective one of the second ribs when the ligation clip is in the clamped position.

### CROSS-REFERENCE TO RELATED APPLICATION

This application claims the benefit of and priority to U.S. Provisional Patent Application No. 62/621,163 filed Jan. 24, 2018, the entire disclosure of which is incorporated by reference herein.

### BACKGROUND

### 1. Technical Description

The present disclosure is directed to ligation clips for sealing body vessels and, more particularly, to ligation clips that include clamping surfaces with tissue retention features for sealing body vessels.

### 2. Background of Related Art

Ligation clips are well known in the surgical arts and are commonly used during a variety of surgical procedures to ligate tissue, e.g., a body vessel. Typically, ligation clips include first and second jaws that include clamping surfaces. The jaws are pivotably connected to each other and movable between open and clamped positions. In use, the ligation clip is clamped to a portion of the body vessel to clamp the body vessel between the clamping surfaces of the first and second jaws. After the ligation clip is clamped about the body vessel, any movement of the ligation clip in relation to the body vessel may have a negative impact on the performance of the ligation clip. This may affect the success of the surgical procedure and threaten the well-being of the patient.

A continuing need exists in the art for ligation clips that have improved retention characteristics to prevent movement of the ligation clip in relation to a body vessel after placement.

### SUMMARY

One aspect of the disclosure is directed to a ligation clip including a first jaw and a second jaw. The first jaw defines a first longitudinal axis and has a first clamping surface supporting first ribs. Each of the first ribs is spaced from each other along the first clamping surface and defines an axis that defines an angle β with the first longitudinal axis. The second jaw has a second clamping surface in opposition to the first clamping surface and defines a second longitudinal axis that is parallel to the first longitudinal axis. The second clamping surface supports second ribs. The second ribs are spaced from each other along the second clamping surface and define an axis that defines the angle β with the second longitudinal axis. The second jaw is pivotably coupled to the first jaw such that the ligation clip is movable between an open position and a clamped position. Each of the first ribs includes a distal wall and a proximal wall and a wall in opposition to the second clamping surface and each of the second ribs includes a distal wall, a proximal wall, and a wall in opposition to the first clamping surface. The distal wall of at least some of the first ribs is positioned in close proximity to the proximal wall of a respective one of the second ribs when the ligation clip is in the clamped position.

In embodiments, the angle β is between about 60 degrees and about 90 degrees.

In some embodiments, the angle β is between about 75 degrees and about 85 degrees.

In certain embodiments, the angle β is about 80 degrees.

In embodiments, the first and second jaws support bosses that are configured to engage a clip applier.

In some embodiments, the ligation clip is formed of a polymeric material.

In certain embodiments, the first jaw includes a first locking element and the second jaw includes a second locking element that is pivotable into engagement with the first locking element to retain the ligation clip in the clamped position.

In embodiments, the first and second jaws are curved along the first and second longitudinal axes.

In some embodiments, the second jaw supports tissue engaging members that are configured to grip and stretch tissue as the ligation clip is moved from the open position towards the clamped position.

In certain embodiments, each of the tissue engaging members includes a distal face that is downwardly sloped in a distal direction.

In embodiments, the first jaw is pivotably coupled to the second jaw by a living hinge.

In some embodiments, the ribs have a rectangular configuration.

In certain embodiments, the walls of the first ribs in opposition to the second clamping surface of the second jaw and the walls of the second ribs in opposition to the first clamping surface of the first jaw are flat.

In embodiments, the walls of the first ribs in opposition to the second clamping surface of the second jaw and the walls of the second ribs in opposition to the first clamping surface of the first jaw are curved.

In some embodiments, the second jaw includes a resilient retaining member that is positioned to be engaged by the first locking element of the first jaw when the ligation clip is moved to the clamped position to retain the ligation clip in the clamped position.

### BRIEF DESCRIPTION OF THE DRAWINGS

Various exemplary embodiments of the presently disclosed ligation clip are described herein below with reference to the drawings, wherein:

FIG. 1 is a side perspective view of an exemplary embodiment of the presently disclosed ligation clip in an open position;

FIG. 2 is an enlarged view of the indicated area of detail shown in FIG. 1;

FIG. 2A is an enlarged top view of a portion of a jaw of the ligation clip shown in FIG. 1;

FIG. 3 is a side perspective view of the ligation clip shown in FIG. 1 in the clamped position;

FIG. 4 is an enlarged view of the indicated area of detail shown in FIG. 3;

FIG. 5 is a side view of the ligation clip shown in FIG. 1 in the open position placed about tissue;

FIG. 6 is a side view of the ligation clip shown in FIG. 5 in the clamped position placed about tissue; and

FIG. 7 is an enlarged view of the indicated area of detail shown in FIG. 6.

### DETAILED DESCRIPTION OF EMBODIMENTS

The presently disclosed ligation clip will now be described in detail with reference to the drawings in which like reference numerals designate identical or corresponding elements in each of the several views. However, it is to be understood that the disclosed embodiments are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

In this description, the term “proximal” is used generally to refer to that portion of the device that is closer to a clinician, while the term “distal” is used generally to refer to that portion of the device that is farther from the clinician. In addition, the term “clinician” is used generally to refer to medical personnel including doctors, nurses, and support personnel. Further, the term “about” is used generally to mean plus or minus 10 percent of the disclosed parameter.

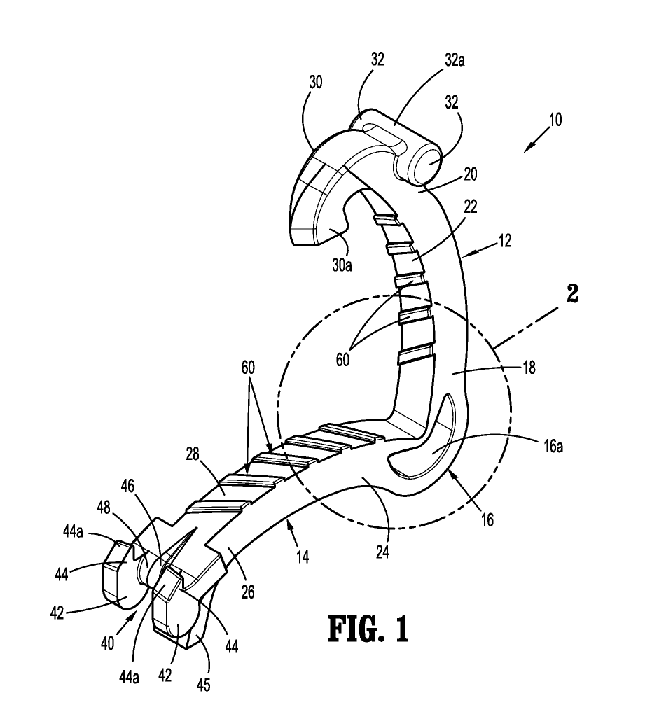

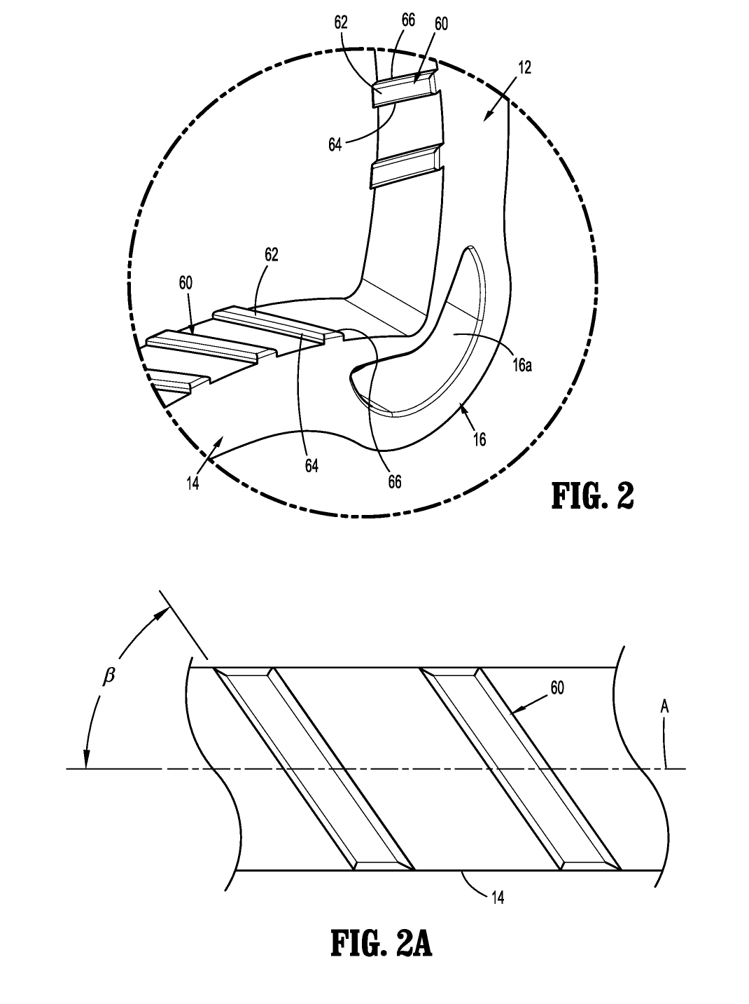

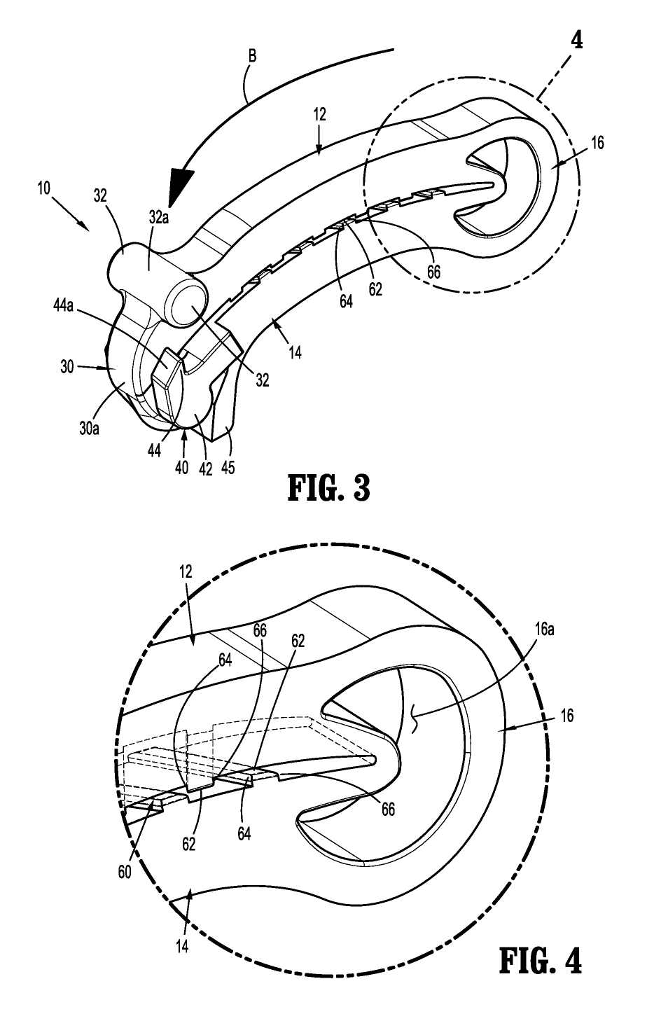

Referring to FIGS. 1-4, an exemplary embodiment of the presently disclosed ligation clip is shown generally as 10. The ligation clip 10 defines a longitudinal axis “A” (FIG. 2A) and includes a first jaw 12, a second jaw 14, and a hinge portion 16 coupling the first jaw 12 to the second jaw 14. The first jaw 12 is pivotable in relation to the second jaw 14 about the hinge portion 16 to move the ligation clip 10 between an open position (FIG. 1) and a clamped position (FIG. 3). In embodiments, the first and second jaws 12, 14 are curved along the longitudinal axis “A” of the ligation clip 10 although other jaw configurations are envisioned. The hinge portion 16 can be integrally formed with the first and second jaws 12, 14, e.g., a living hinge, and may define a crescent shaped through bore 16a to facilitate movement of the first jaw 12 in relation to the second jaw 14 between the open and clamped positions. The through bore 16a also allows for substantially complete closure of the first and second jaws 12, 14 adjacent a proximal end of the jaws 12, 14.

The first jaw 12 defines a longitudinal axis and includes a proximal portion 18, a distal portion 20, and a clamping surface 22. The second jaw 14 defines a longitudinal axis that is parallel to the longitudinal axis of the first jaw 12 and includes a proximal portion 24, a distal portion 26, and a clamping surface 28. The proximal portions 18, 24, of the first and second jaws 12, 14, respectively, are coupled to the hinge portion 16.

The distal portion 20 of first jaw 12 includes a first locking element 30 and spaced bosses 32. The first locking element 30 has a hooked portion 30a that extends downwardly and proximally from the tissue clamping surface 22 to define a portion of a latching mechanism (FIG. 6) that is configured to retain the ligation clip 10 in a clamped position as described in further detail below. The bosses 32 are positioned and configured to engage the jaws of an applicator (not shown) to facilitate application of the ligation clip 10 to tissue. In embodiments, the bosses 32 may be form a portion of a single cylindrical member 32a.

The distal portion 26 of the second jaw 14 includes a second locking element 40, spaced bosses 42, tissue engaging members 44, and a resilient retaining member 45. The resilient retaining member 45 is positioned to be engaged by the first locking element 30 of the first jaw 12 when the ligation clip 10 is moved to the clamped position such that the retaining member is biased into the first locking element 30 to urge the first locking element 30 in relation to the second locking element 40 to the latched position (FIG. 6). The second locking element 40 defines a recess 46 and a cam surface 48. The cam surface 48 and the recess 46 are configured to receive and guide the first locking element 30 into locking engagement with the second locking element 40 when the ligation clip 10 is moved to the clamped position (FIG. 3) to secure the ligation clip 10 in the clamped position. In particular, the cam surface 48 is configured to deflect the first locking element 30 outwardly in a distal direction as the first locking element 30 passes over the cam surface 48 such that when the first locking element 30 moves past the cam surface 48, the first locking element 30 resiliently moves inwardly into engagement with the second locking element 40 to retain the ligation clip 10 in the clamped position.

In embodiments, each of the tissue engaging members 44 has a downwardly sloped distal face 44a that is configured to grip and stretch tissue as the ligation clip 10 is moved from the open position (FIG. 1) towards the clamped position (FIG. 3) to improve the ligating characteristics of the ligation clip 10. The bosses 42 are similar to the bosses 32 of the first jaw 12 and are configured to engage jaws of an applicator (not shown) to facilitate application of the ligation clip 10 to tissue.

The first and second clamping surfaces 22, 28 of the first and second jaws 12, 14, respectively, are substantially flat and support a plurality of diagonal ribs 60. Each of the ribs 60 defines an angle β (FIG. 2A) with the longitudinal axis “Z” of the ligation clip 10. In embodiments, the angle β is between about 60 degrees and about 90 degrees. In other embodiments, the angle β is between about 75 degrees and about 85 degrees. In other embodiments, the angle β is between about 80 degrees.

In embodiments, each of the ribs 60 has a substantially rectangular configuration including a wall 62 in opposition to the tissue clamping surface 22, 28 of the respective opposing jaw 12, 14, and substantially vertical distal and proximal walls 64 and 66, respectively. In embodiments, the opposing wall 62 is substantially flat and the distal and proximal walls 64, 66 have a small degree of slope. Alternately, the proximal and distal walls can be vertical of include more of a slope and the opposing wall 62 need not be flat but rather may be curved or include tissue engaging structure such as ribs, knurls or the like. In some embodiments, each or some of the ribs 60 extend entirely across the clamping surface 22, 28 of the respective first or second jaw 12, 14. Alternately, some or all of the ribs 60 on one or both of the clamping surfaces 22, 28 may only extend partly across the clamping surface 22, 28 of the respective first or second jaw 12, 14.

Each of the ribs 60 of the first jaw 12 includes a longitudinal axis that is substantially parallel to the longitudinal axis of the ribs 60 supported on the second jaw 14. The size and axial spacing of the ribs 60 on each of the clamping surfaces 22, 28 of the first and second jaws 28 may vary.

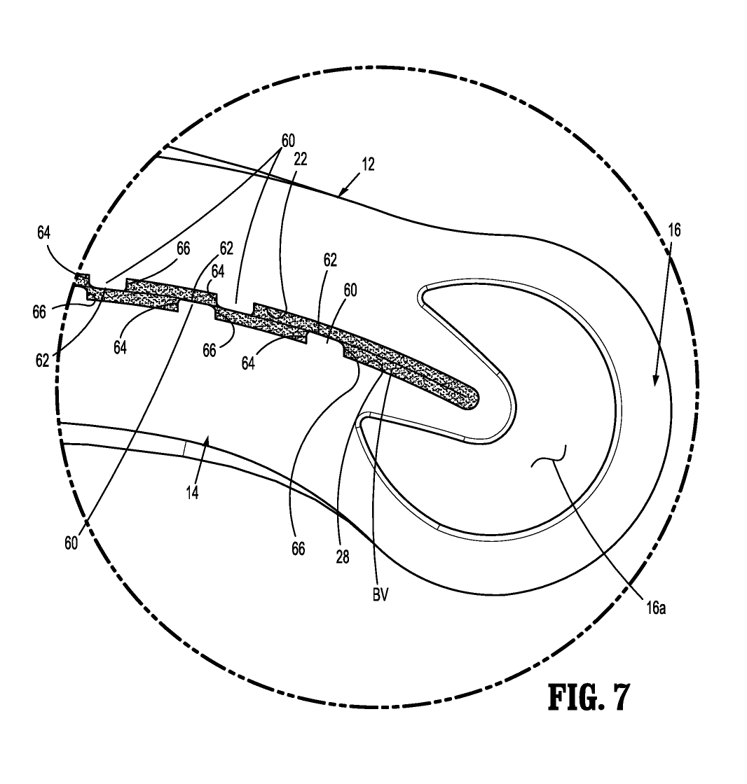

In embodiments, the distal wall 64 of the ribs 60 on the first jaw 12 is positioned to be in substantial alignment with the proximal wall 66 of the ribs 60 on the second jaw 14 when the ligation clip 10 is in the clamped position. As used herein, “substantial alignment” means that the distal wall 64 of the ribs 60 on the first jaw 12 are positioned in close proximity with a respective one of the proximal walls 66 of the ribs 60 on the second jaw 14 such that the distal wall 64 is positioned in full alignment, offset slightly distally, or offset slightly proximally than the proximal wall 66 of the ribs 60 of the second jaw 14. In some embodiments, the opposing walls 62 of the ribs 60 on opposing jaws 12, 14 are axially spaced and do not overlap when the ligation clip 10 is in the clamped position. With the ribs 60 positioned in this manner, a body vessel “By” (FIG. 7) supported between the jaws 12, 14 is compressed between the opposing walls 62 of the ribs and the clamping surfaces 22, 28 of the first and second jaws 12, 14, and between the proximal and distal walls 62, 64 of the ribs 60 on the clamping surfaces 22, 28 of the first and second jaws 12, 14, respectively. This combined clamping action improves the retention capabilities of the ligation clip 10 on the body vessel “By”.

Referring to FIGS. 3 and 4, when the first jaw 12 and the second jaw 14 are moved from the open position (FIG. 1) to the clamped position (FIG. 3) in the direction indicated by arrow “B” in FIG. 3, the first jaw 12 pivots in relation to the second jaw 14 about the hinge portion 16 to move the hooked portion 30a of the first locking element 30 into engagement with the second locking element 40 to secure the ligation clip 10 in the clamped position. In the clamped position, the distal walls of the ribs 60 on the clamping surface 22 of the first jaw 12 are positioned in close alignment and proximity to the proximal walls of the ribs 60 formed on the second jaw 14 as described above.

In embodiments, the surgical ligation clip 10 may be comprised of a resilient bioabsorbable and/or biocompatible polymeric material. Examples of suitable bioabsorbable and/or biocompatible polymers include acetal polyoxymethylene (POM), polyethylene terephthalate (PET), polybutylene terephthalate (PBT), polyoxymethylene, polyetheretherketone (PEEK), polypropylene, and polyethylene or other thermoplastic materials having similar properties that can be injection-molded. The ligation clip 10 may also be comprised of a polymeric material or materials in combination with radiolucent metal alloys. Alternately, other materials may be used to form the ligation clip 10 including biocompatible metals, plastics and composites.

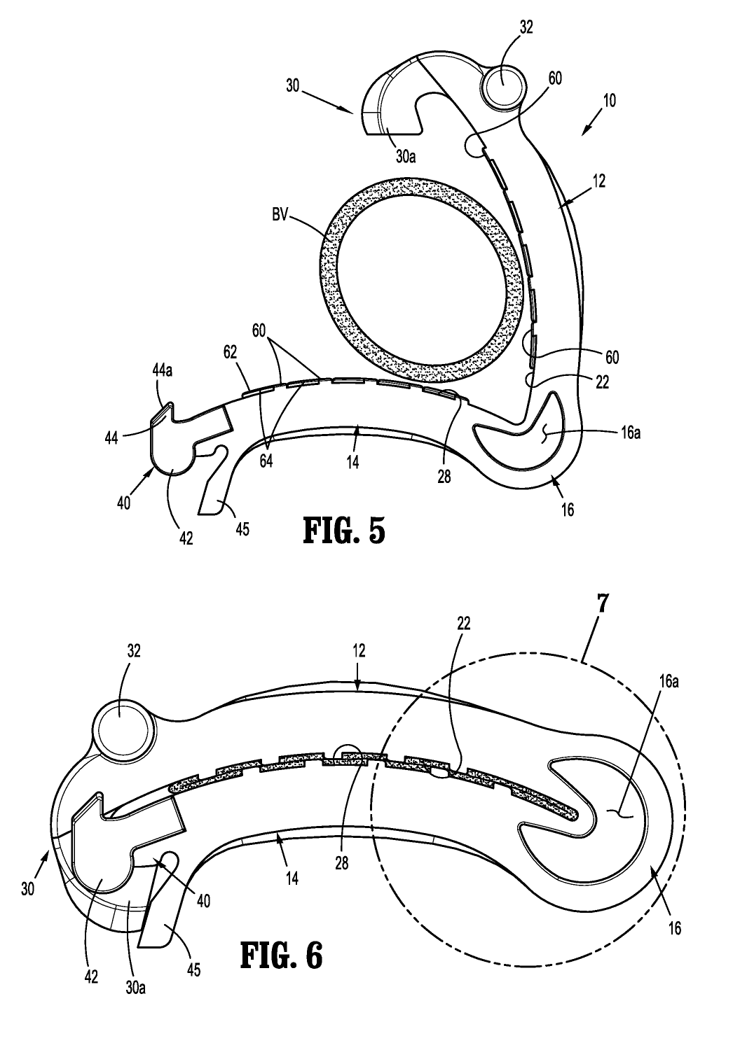

Referring to FIGS. 5-7, in use, the ligation clip 10 is positioned about tissue, e.g., a body vessel “By”, such that the body vessel “BV” is positioned between the tissue clamping surface 22 of the first jaw 12 and the tissue clamping surface 28 of the second jaw 14. As described above, when the ligation clip 10 is moved from the open position (FIG. 1) to the closed position (FIG. 6), the first jaw 12 pivots in relation to the second jaw 14 about the hinge 16 to move the hooked portion 30a of the first locking element 30 into engagement with the second locking element 40 to secure the ligation clip 10 in the clamped position.

As shown in FIGS. 6 and 7, in the clamped position, the body vessel “By” is compressed between the first and second clamping surfaces 22, 28 of the first and second jaws 12, 14, respectively. More particularly, when the ligation clip 10 is moved to the clamped position, the body vessel “By” is compressed between the clamping surfaces 22, 28 of the first and second jaws 12, 14, respectively. In addition, portions of the body vessel “BV” are compressed between the opposing walls 62 of the ribs 60 and the clamping surfaces 22, 28 of the first and second jaws 12, 14, respectively, and between the distal walls 64 of the ribs 60 of the first jaw 12 and the proximal walls 66 of the ribs 60 of the second jaw 14. As described above, this combination of compression forces improves retention of the ligation clip 10 about a body vessel “BV” and minimizes the likelihood that the ligation clip 10 will move in relation to the body vessel “BV” after placement of the ligation clip 10.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

1. A ligation clip defining a longitudinal axis and comprising:
a first jaw defining having a first clamping surface, the first clamping surface supporting first ribs, each of the first ribs being axially spaced from each other along the first clamping surface and defining an axis that defines an angle β with the longitudinal axis of the ligation clip; and
a second jaw having a second clamping surface in opposition to the first clamping surface, the second clamping surface supporting second ribs, each of the second ribs being spaced from each other along the second clamping surface and defining an axis that defines the angle β with the longitudinal axis of the ligation clip, the second jaw being pivotably coupled to the first jaw such that the ligation clip is movable between an open position in which the first clamping surface is spaced from the second clamping surface and a clamped position in which the first clamping surface is in juxtaposed alignment with the second clamping surface.
wherein each of the first ribs includes a distal wall and a proximal wall and a wall in opposition to the second clamping surface and each of the second ribs includes a distal wall, a proximal wall, and a wall in opposition to the first clamping surface, the distal wall of at least some of the first ribs being positioned in substantial alignment with the proximal wall of a respective one of the second ribs when the ligation clip is in the clamped position.

2. The ligation clip of claim 1, wherein the angle β is between about 60 degrees and about 90 degrees.

3. The ligation clip of claim 2, wherein the angle β is between about 75 degrees and about 85 degrees.

4. The ligation clip of claim 3, wherein the angle β is about 80 degrees.

5. The ligation clip of claim 1, wherein the first and second jaws support bosses, the bosses being configured to engage a clip applier.

6. The ligation clip of claim 1, wherein the ligation clip is formed of a polymeric material.

7. The ligation clip of claim 1, wherein the first jaw includes a first locking element and the second jaw includes a second locking element, the second locking element being pivotable into engagement with the first locking element to retain the ligation clip in the clamped position.

8. The ligation clip of claim 1, wherein the first and second jaws are curved along the longitudinal axis of the ligation clip.

9. The ligation clip of claim 2, wherein the second jaw supports tissue engaging members that are configured to grip and stretch tissue as the ligation clip is moved from the open position towards the clamped position.

10. The ligation clip of claim 9, wherein each of the tissue engaging members includes a distal face that is downwardly sloped in a distal direction.

11. The ligation clip of claim 1, wherein the first jaw is pivotably coupled to the second jaw by a living hinge.

12. The ligation clip of claim 1, wherein the first and second ribs have a rectangular configuration.

13. The ligation clip of claim 1, wherein the walls of the first ribs in opposition to the second clamping surface of the second jaw and the walls of the second ribs in opposition to the first clamping surface of the first jaw are flat.

14. The ligation clip of claim 1, wherein the walls of the first ribs in opposition to the second clamping surface of the second jaw and the walls of the second ribs in opposition to the first clamping surface of the first jaw are curved.

15. The ligation clip of claim 7, wherein the second jaw includes a resilient retaining member, the resilient retaining member being positioned to be engaged by the first locking element of the first jaw when the ligation clip is moved to the clamped position to retain the ligation clip in the clamped position.

16. A ligation clip defining a longitudinal axis and comprising:
a first jaw defining having a first clamping surface, the first clamping surface supporting first ribs, each of the first ribs being axially spaced from each other along the first clamping surface; and
a second jaw having a second clamping surface in opposition to the first clamping surface, the second clamping surface supporting second ribs, each of the second ribs being spaced from each other along the second clamping surface, the second jaw being pivotably coupled to the first jaw such that the ligation clip is movable between an open position in which the first clamping surface is spaced from the second clamping surface and a clamped position in which the first clamping surface is in juxtaposed alignment with the second clamping surface;
wherein each of the first and second ribs includes a distal wall and a proximal wall , the distal wall of each of the first ribs being positioned in substantial alignment with the proximal wall of a respective one of second ribs when the ligation clip is in the clamped position.

17. The ligation clip of claim 16, wherein the ligation clip is formed of a polymeric material.

18. The ligation clip of claim 16, wherein the first and second ribs have a rectangular configuration.

19. A ligation clip comprising:
a first jaw defining having a first clamping surface, the first clamping surface supporting first ribs, each of the first ribs being axially spaced from each other along the first clamping surface; and
a second jaw having a second clamping surface in opposition to the first clamping surface, the second clamping surface supporting second ribs, each of the second ribs being spaced from each other along the second clamping surface, the second jaw being pivotably coupled to the first jaw such that the ligation clip is movable between an open position in which the first clamping surface is spaced from the second clamping surface and a clamped position in which the first clamping surface is in juxtaposed alignment with the second clamping surface;
wherein each of the first ribs includes a distal wall and each of the second ribs includes a proximal wall, the distal wall of each of the first ribs being positioned in substantial alignment with the proximal wall of a respective one of the second ribs when the ligation clip is in the clamped position.

20. The ligation clip of claim 19, wherein the ligation clip defines a longitudinal axis, the first ribs being axially spaced from each other along the first clamping surface and defining an axis that defines an angle β with the longitudinal axis of the ligation clip, and the second ribs being axially spaced from each other along the second clamping surface and defining an axis that defines the angle β with the longitudinal axis of the ligation clip.
